(19) United States
(12) Patent Application Publication
(10) Pub. No.: US 2024/0331172 A1
(43) Pub. Date: Oct. 3, 2024

(54) EYE TRACKER WITH MULTIPLE CAMERAS

(71) Applicant: ALCON INC., Fribourg (CH)

(72) Inventors: Mario Abraham, Burgthann (DE); Maik Lange, Ruckersdork (DE)

(21) Appl. No.: 18/614,824

(22) Filed: Mar. 25, 2024

Related U.S. Application Data
(60) Provisional application No. 63/492,639, filed on Mar. 28, 2023.

(51) Int. Cl. G06T 7/292; A61B 3/113; A61B 3/14; A61F 9/008; G06T 7/246

(52) U.S. Cl. CPC G06T 7/292; A61B 3/113; A61B 3/14; A61F 9/008; G06T 7/246; A61F 2009/00846; G06T 2207/10021; G06T 2207/10048; G06T 2207/20212; G06T 2207/30041

(57) ABSTRACT

In certain embodiments, an ophthalmic system tracks movement of an eye region and includes a camera system and a computer. The camera system has cameras that yield image portions of the eye region, where each camera images at least a part of the eye region. The camera system has a system axis and system field of view. The eye region includes one or both eyes, and each eye has an eye center and eye axis. The computer receives the image portions from the camera system and tracks movement of at least one eye according to the image portions.

### TECHNICAL FIELD

The present disclosure relates generally to ophthalmic systems, and more particularly to an eye tracker with multiple cameras.

### BACKGROUND

Certain ophthalmic systems utilize an eye tracker to monitor movement of the eye. For example, in laser-assisted in situ keratomileusis (LASIK) surgery, laser pulses are directed towards the eye in a particular pattern to ablate tissue to reshape the cornea. To effectively treat the eye, the laser beam should be accurately directed to specific points of the eye-even as the eye moves. Accordingly, an eye tracker is used to monitor movement of the eye.

### BRIEF SUMMARY

In certain embodiments, an ophthalmic system tracks movement of an eye region and includes a camera system and a computer. The camera system has cameras that yield image portions of the eye region, where each camera images at least a part of the eye region. The camera system has a system axis and field of view. The eye region includes one or both eyes, and each eye has an eye center and axis. The computer receives the image portions from the camera system and tracks movement of at least one eye according to the image portions.

Embodiments may include none, one, some, or all of the following features:
The computer tracks the movement of at least one eye in two dimensions.
The computer tracks the movement of at least one eye in three dimensions to allow for 6D tracking.
The cameras includes a set of stereoscopic cameras arranged symmetrically about the system axis.
The cameras includes a coaxial camera aligned with the system axis.
The cameras includes an asymmetrically arranged camera that lacks a corresponding camera symmetrical about the system axis.
The cameras include a higher speed camera that generates images at greater than 400 frames per second.
The cameras include a higher resolution camera that generates images with greater than 4 megapixels.
At least one camera detects a range of visible light from the eye region to yield an image portion.
At least one camera detects a range of infrared light from the eye region to yield an image portion.
At least one camera detects a range of ultraviolet light from the eye region to yield an image portion.
A light projector directs a pattern of light towards at least one eye of the eye region. At least one camera detects the pattern of light reflected by the at least one eye.
The computer aligns the image portions to yield a combined image of the eye region and tracks movement of the at least one eye according to the combined image.

In certain embodiments, a method for tracking movement of an eye region includes providing, by a camera system of cameras, image portions of the eye region. The camera system has cameras that yield image portions of the eye region, where each camera images at least a part of the eye region. The camera system has a system axis and system field of view. The eye region includes one or both eyes, and each eye has an eye center and eye axis. A computer receives the image portions from the camera system and tracks movement of at least one eye of the eye region according to the image portions.

Embodiments may include none, one, some, or all of the following features:
The method further includes tracking the movement of at least one eye in two dimensions.
The method further includes tracking the movement of at least one eye in three dimensions to allow for 6D tracking.
The method further includes generating images at greater than 400 frames per second.
The method further includes generating images with greater than 4 megapixels.
The method further includes directing, by a light projector, a pattern of light towards at least one eye. At least one camera detects the pattern of light reflected by the eye.

### BRIEF DESCRIPTION OF THE DRAWINGS

FIG. 1 illustrates an example of an ophthalmic system with an eye tracker, according to certain embodiments;

FIGS. 2A and 2B illustrate an example of the field of view (FOV) of a camera system of FIG. 1, according to certain embodiments;

FIGS. 3A and 3B illustrate examples the camera system of FIG. 1 tracking example eye regions, according to certain embodiments;

FIG. 4 illustrates an example of a stereoscopic arrangement of cameras of the camera system of FIG. 1, according to certain embodiments;

FIG. 5 illustrates an example of a stereoscopic and coaxial arrangement of the camera system of FIG. 1, according to certain embodiments;

FIG. 6 illustrates an example of an asymmetrical arrangement of cameras of the camera system of FIG. 1, according to certain embodiments; and

FIG. 7 illustrates an example of a method that may be performed by the ophthalmic system of FIG. 1, according to certain embodiments.

### DESCRIPTION OF EXAMPLE EMBODIMENTS

Referring now to the description and drawings, example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

In certain eye trackers, a light projector directs light towards the eye at a known angle, and a camera generates images that show the light reflections on the eye. Assumptions based on a standard eye model are used to determine the movement of the eye from the camera images. The assumptions, however, may not accurately describe the particular patient's eye, rendering the tracking less accurate.

The eye trackers described herein do not require eye model assumptions, so may provide more accurate tracking. The eye trackers include a camera system with cameras that image the eye from different directions, e.g., coaxially and obliquely. From the known positions of the cameras, eye movement can be determined from the resulting images. The trackers can track, e.g., translational and/or rotational movement in the x, y, and/or z directions. In certain embodiments, the cameras may record infrared (IR), visible light, and/or other light, and may record images at a higher speed and/or a higher resolution. The eye trackers may be used in ophthalmic diagnostic and/or treatment systems (e.g., in refractive or cataract surgery).

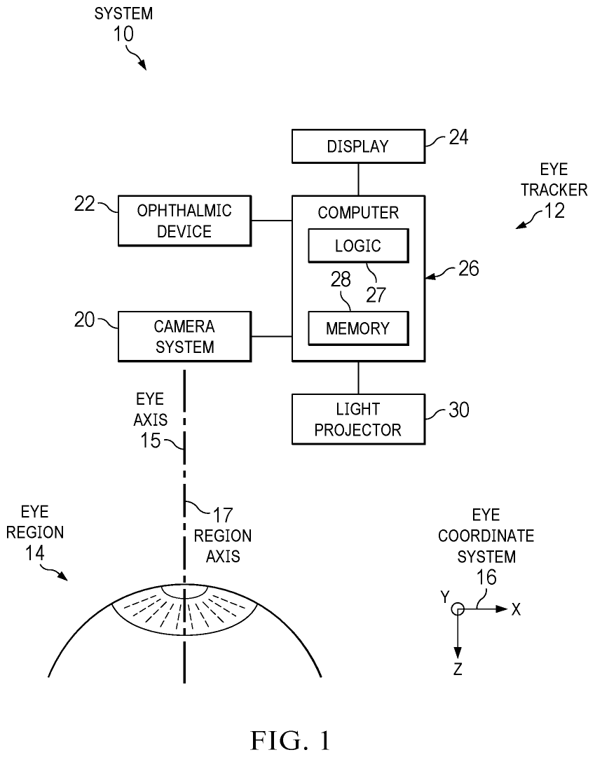

FIG. 1 illustrates an example of an ophthalmic system 10 with an eye tracker 12 that monitors an eye region 14 that includes one or both eyes of a patient, according to certain embodiments. In general, eye tracker 12 monitors the movement of one or more features of the eye (e.g., pupil, iris, blood vessels, limbus, sclera, eyelashes, and/or eyelid) in images to track the movement of the eye.

For case of explanation, certain eye features are used to define an example coordinate system 16 (x, y, z) of the eye. For example, the eye has a center (e.g., pupil center, apex, vertex) and an eye axis 15 (e.g., optical or pupillary axis) that can define the z-axis of eye coordinate system 16, which in turn defines an xy-plane of system 16. Eye region 14 has a region axis 17. If eye region 14 has one eye, region axis 17 may substantially coincide with eye axis 15. If eye region 14 has two eyes, region axis 17 may pass through a midpoint between the eyes.

As an overview of the example system, ophthalmic system 10 includes an eye tracker 12, an ophthalmic device 22, a display 24, and a computer 26 (which includes logic 27 and memory 28), coupled as shown. Eye tracker 12 includes a camera system 20, and computer 26, coupled as shown. In certain embodiments, eye tracker 13 includes a light projector 30 to allow for tracking in the z-direction. As an example of an overview of operation, camera system 20 of eye tracker 12 has cameras that yield image portions of eye region 14. Each camera is located at a known position (e.g., a known location and/or orientation relative to each other and/or to eye region 14) and records at least a portion of eye region 14 to yield an image portion. As described in more detail below, the known positions allow for calculation of eye movement. Computer 26 receives the image portions from camera system 20 and tracks the movement of at least one eye according to the image portions.

Turning to the components of the example, eye tracker 12 may track movement of an eye in six “dimensions” (6D), i.e., “6D tracking”. The six dimensions include x-translational, y-translational, z-translational, rotational, x-rolling, and/or y-rolling movements, relative to eye coordinate system 16. In certain embodiments, x-, y-, and z-translational movement may be translational movement in the x-, y-, and z-directions, respectively. Rotational movement may be movement about eye axis 15. X- and y-rolling movements may be rotational movement about the x- and y-axes, respectively. In particular embodiments, 6D tracking may track some or all of the 6D movements.

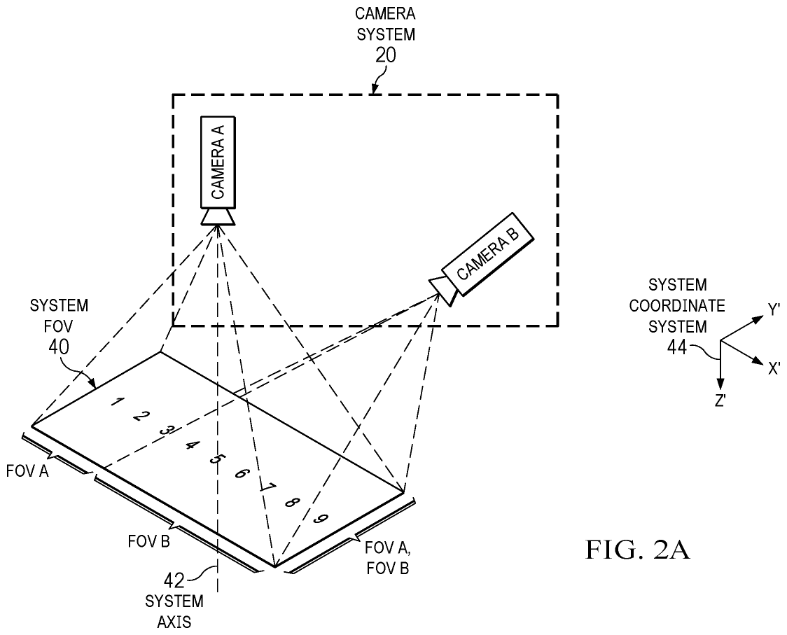

In certain embodiments, eye tracker 12 includes camera system 20 that generates images of eye region 14. Camera system 20 has a field of view (FOV) (described in more detail with respect to FIGS. 2A and 2B) that covers eye region 14. The FOV has a known relationship to the coordinate system of camera system 20, which in turn has a known relationship to the coordinate system that ophthalmic device 22 uses to treat and/or diagnose an eye. Eye tracker 12 tracks the movement of an eye by tracking the movement of the eye relative to the FOV. The eye tracking information may be used by ophthalmic device 22 to treat and/or diagnose the eye.

In the embodiments, camera system 20 includes cameras. For case of explanation, the “position” of a camera relative to eye region 14 may describe the distance between the camera and eye region 14 and the direction of the camera axis relative to region axis 17. A camera detects light from an object and generates a signal in response to the light. The signal carries image data that can be used to generate the image of the eye. The image data are provided to computer 26 for eye tracking (and optionally other analysis) and may also be provided to display 24 to present the images of the eye. Examples of cameras include a charged-coupled device (CCD), video, complementary metal-oxide semiconductor (CMOS) sensor (e.g., active-pixel sensor (APS)), line sensor, and optical coherence tomography (OCT) camera.

A camera detects light of any suitable spectral range, e.g., a range of infrared (IR), ultraviolet (UV), and/or visible (VIS) wavelength light, where a range can include a portion or all of the wavelength. For example, a camera may detect visible light, infrared light, or other visible and infrared light from eye region 14 to yield an image portion. Certain cameras may capture features of the eye (e.g., pupil, iris structures, blood vessels, limbus, etc.) better than others. For example, an infrared camera generally provides more stable pupil tracking and better contrast for iris structures. Accordingly, an IR camera may be used to monitor lateral movement by tracking the pupil and/or cyclotorsion by tracking iris structures. As another example, a visible range camera yields better images of blood vessels, so a visible range camera may be used to monitor translation and/or rotational movement by tracking blood vessels.

A camera may record images at any suitable frequency or resolution. A higher speed camera may record images at greater than, e.g., 400 to 1500 frames per second, such as greater than 500, 750, or 1000 frames per second. A higher resolution camera may yield images with greater than, e.g., 4 to 24 megapixels, such as greater than 5, 10, 15, or 20 megapixels. In general, higher resolution images and higher speed image acquisition may provide more accurate tracking, but both features may require more computing time, so there may be a trade-off between resolution and speed. Accordingly, the speed and/or resolution of a camera may be selected for particular purposes. In certain embodiments, a higher speed camera may track eye features that move faster and/or can be identified with lower resolution, and a higher resolution camera may be used to track eye features that require higher resolution for identification and/or move more slowly. For example, a lower resolution, higher speed camera may track the pupil (which does not require high resolution) to detect xy-movement. As another example, a higher resolution, lower speed camera may track blood vessels/iris structures to detect rotations, z-movement.

Ophthalmic device 22 may be a system that is used to diagnose and/or treat an eye. Examples include a refractive surgical system, a cataract system, a topographer, an OCT measuring device, and a wavefront measuring device. Display 24 provides images, e.g., the image portions and/or the combined image, to the user of system 10. Examples of display 24 include a computer monitor, a 3D display, a projector/beamer, a TV monitor, binocular displays, glasses with monitors, a virtual reality display, an augmented reality, and a mixed reality display.

Light projector 30 directs a pattern of light towards eye region 14, and the reflection of the light is used to track the eye. Light projector 30 may comprise one or more light sources that yield the pattern of light. The light projections may be used in any suitable manner. For example, the light may be directed at a known angle, which can be used to align the image portions. As another example, the curvature of the eye distorts line projections, so the line distortions may help identify the border between the cornea and sclera where the curvature changes. As yet another example, a symmetric projection may be used to identify the vertex or apex of the eye. As yet another example, a stripe projector may project lines at an angle to the eye, so the lines appear curved at the cornea and change in curvature as the eye moves. Any suitable pattern may be used, e.g., a line (such as a stripe), a cross, and/or an array of lines and/or dots.

Computer 26 controls components of system 10 (e.g., camera system 20, an ophthalmic device 22, a display 24, and/or light projector 30) to track an eye. In the example, computer 16 receives the image portions from camera system 20 and tracks the movement of at least one eye according to the image portions. In certain embodiments, computer 26 aligns the image portions to yield a combined image of eye region 14 and tracks the movement of at least one eye according to the combined image.

FIGS. 2A and 2B illustrate an example of the field of view (FOV) 40 of camera system 20 of FIG. 1, according to certain embodiments. A camera of camera system 20 has a field of view (FOV) that detects light from eye region 14 to yield an image portion 45 of some or all of eye region 14. Different cameras can have different FOVs that detect light from different portions of eye region at different directions, and different FOVs may overlap. The combined FOVs from the cameras yield a system FOV 40. In general, more cameras at different positions (locations and orientations) may improve the detection of eye features and the accuracy of the tracking.

In the example, camera system 20 has a system FOV 40, a system axis 42, and a system coordinate system 44 (x′, y′, z′). System axis 42 may have any suitable position, e.g., axis 42 may be substantially orthogonal to system FOV 40 and may pass through the center of system FOV 40. System axis 42 and system coordinate system 44 (x′, y′, z′) may be related in any suitable manner. In the example, system axis 42 defines the z′-axis of system coordinate system 44. In the example, system FOV 40 is generally planar and images the numbers 1 through 9. Camera system 20 includes Camera A with FOV A and Camera B with FOV B. FOV A covers system FOV 40 (i.e., images numbers 1 through 9), and FOV B covers only part of system FOV 40 (i.e., images numbers 4 through 9). Camera A yields image portion A, and Camera B yields image portion B.

In certain embodiments, computer 26 aligns and combines image portions 45 to yield combined image 46. Image portions 45 may be aligned in any suitable manner. For example, each camera has a known position, such as a location (e.g., distance away from system FOV 40 and/or eye region 14) and orientation (e.g., camera optical axis relative to system axis 42 and/or eye axis 15, or viewing angle), as well as dimensions and imaging properties. From this information, computer 26 can determine the positions of image portions 45 to align them within combined image 46. As another example, the cameras each generate an image of a calibration figure (e.g., a checkerboard), and the positions of the cameras are determined from the images. As yet another example, a user calibrates image portions 45 by manually aligning portions 45 when viewed through the cameras. Computer 26 records the positions of the aligned portions.

Image portions 45 may be combined in any suitable manner. For example, image portions 45 may be combined to yield a two-dimensional (2D) image to allow for 2D tracking, and/or image portions 45 (e.g., from stereoscopic cameras) may be combined to yield a three-dimensional (3D) image to allow for 3D tracking.

Eye tracker 12 tracks one or both eyes of eye region 14 according to the image portions and/or combined image 46. For example, computer 26 identifies a target eye feature (e.g., pupil, iris structure, or blood vessel) in the uncombined or combined image portions, and tracks movement of the feature relative to system FOV 40 to track the eye. Computer 26 may identify a feature using an image portion 45 from a camera more likely to produce a better-quality image of the feature. E.g., a camera may have a FOV, wavelength, resolution, and/or speed that is more likely to image the feature. Examples of cameras with such properties imaging particular features are presented throughout this description.

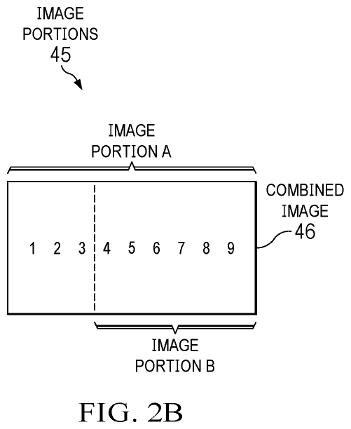

FIGS. 3A and 3B illustrate examples camera system 20 of FIG. 1 tracking eye regions 14, according to certain embodiments. In FIG. 3A, eye region 14 includes one eye. In the example, eye axis 15 of the eye may at first be substantially aligned with system axis 42 of camera system 20. As the eye moves relative to camera system 20, eye axis 15 moves relative to system axis 42.

In FIG. 3B, eye region 14 includes both eyes. System axis 42 of camera system 20 is substantially aligned with the midpoint between the eyes. Camera system 20 includes cameras that image one or both eyes to yield image portions and/or a combined image that images both eyes simultaneously, so camera system 20 can track both eyes simultaneously and independently of one another. In certain embodiments, camera system 20 includes a pair of stereoscopic cameras that can each image both eyes to provide three-dimensional image information, including z-depth information for both eyes.

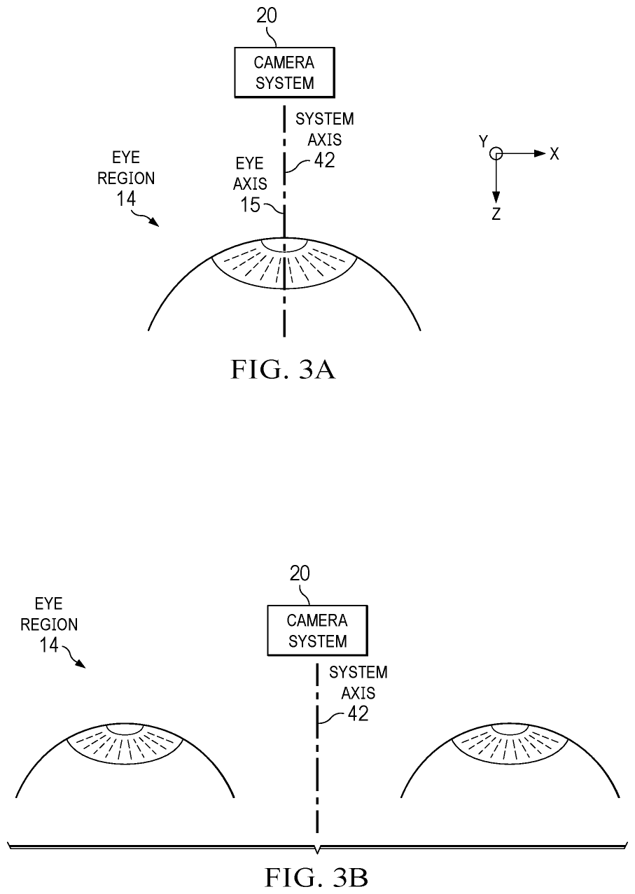

FIG. 4 illustrates an example of a stereoscopic arrangement of cameras of camera system 20a, according to certain embodiments. Camera A-L and Camera A-R are arranged with mirror symmetry about system axis 14, i.e., spatially separated with equal viewing angles on opposite sides of system axis 14. The images may be stereoscopically reconstructed to track the location and the orientation of an eye in three dimensions. The greater the angle and/or distance between the cameras, the better the accuracy in the z-direction. This may facilitate positioning the head of the patient.

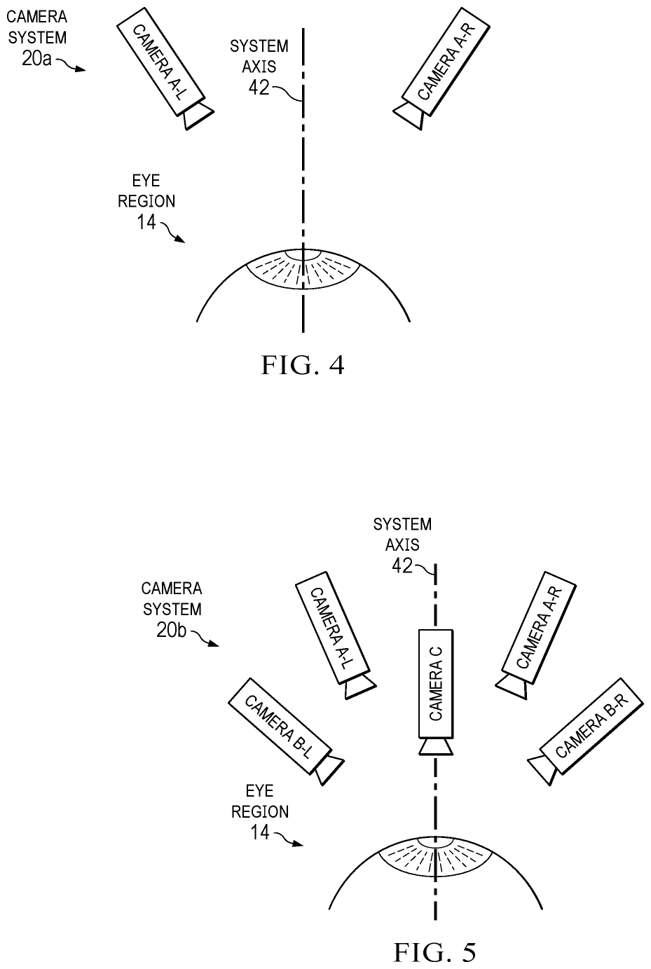

FIG. 5 illustrates an example of a stereoscopic and coaxial arrangement of cameras of camera system 20b, according to certain embodiments. Camera A-L and Camera A-R are stereoscopically arranged, and Camera B-L and Camera B-R are also stereoscopically arranged. Camera C is coaxially arranged, i.e., aligned with system axis 14.

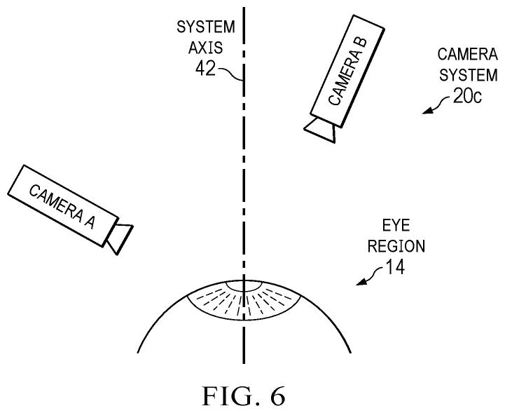

FIG. 6 illustrates an example of an asymmetrical arrangement of cameras of camera system 20c, according to certain embodiments. Camera A and Camera B are asymmetrically arranged at different viewing angles, i.e., the cameras are not mirror symmetric relative to system axis 14. An asymmetrically arranged camera lacks a corresponding camera symmetrical about system axis 14. In the example, neither Camera A nor Camera B has a corresponding camera symmetrical about system axis 14, so they are asymmetric cameras.

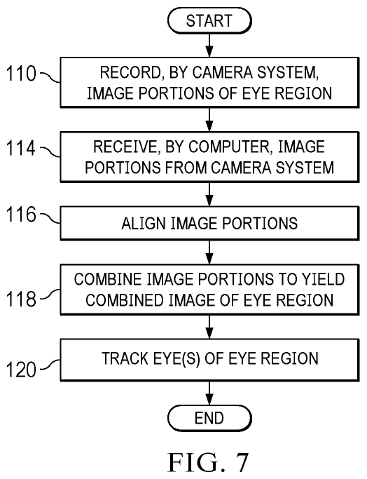

FIG. 7 illustrates an example of a method that may be performed by ophthalmic system 10 of FIG. 1, according to certain embodiments. The method starts at step 110, where camera system 20 records image portions 45 of eye region 14. Image portions may show features of the eye and in some embodiments may show light patterns projected onto the eye.

Computer 26 receives image portions 45 from camera system 20 at step 114. Computer 26 aligns image portions 45 at step 116. For example, computer 26 may determine the relative positions of the image portions from the positions of the cameras, from images of a calibration figure, or from user calibration. In certain embodiments, computer 26 combines the aligned image portions 45 at step 118 to yield a combined image 46 of eye region 14. Combined image 46 may be a two-dimensional (2D) image for tracking in two dimensions or a three-dimensional (3D) image for tracking in three dimensions, which may allow for 6D tracking.

At step 120, computer 26 tracks one or both eyes of eye region 14 according to the image portions and/or combined image 46. The eye(s) may be tracked in any suitable manner. For example, computer 26 may identify a target eye feature in image portions and/or combined image 46 and track movement of the feature to track the eye. As another example, computer 26 may track a particular feature using an image portion 45 from a camera more likely to produce a better-quality image of the feature, e.g., an image generated with higher speed, higher resolution, infrared light, or visible light. The method then ends.

A component (such as the control computer) of the systems and apparatuses disclosed herein may include an interface, logic, and/or memory, any of which may include computer hardware and/or software. An interface can receive input to the component and/or send output from the component, and is typically used to exchange information between, e.g., software, hardware, peripheral devices, users, and combinations of these. A user interface is a type of interface that a user can utilize to communicate with (e.g., send input to and/or receive output from) a computer. Examples of user interfaces include a display, Graphical User Interface (GUI), touchscreen, keyboard, mouse, gesture sensor, microphone, and speakers.

Logic can perform operations of the component. Logic may include one or more electronic devices that process data, e.g., execute instructions to generate output from input. Examples of such an electronic device include a computer, processor, microprocessor (e.g., a Central Processing Unit (CPU)), and computer chip. Logic may include computer software that encodes instructions capable of being executed by an electronic device to perform operations. Examples of computer software include a computer program, application, and operating system.

A memory can store information and may comprise tangible, computer-readable, and/or computer-executable storage medium. Examples of memory include computer memory (e.g., Random Access Memory (RAM) or Read Only Memory (ROM)), mass storage media (e.g., a hard disk), removable storage media (e.g., a Compact Disk (CD) or Digital Video or Versatile Disk (DVD)), database, network storage (e.g., a server), and/or other computer-readable media. Particular embodiments may be directed to memory encoded with computer software.

Although this disclosure has been described in terms of certain embodiments, modifications (such as changes, substitutions, additions, omissions, and/or other modifications) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, or the operations of the systems and apparatuses may be performed by more, fewer, or other components, as apparent to those skilled in the art. As another example, modifications may be made to the methods disclosed herein. The methods may include more, fewer, or other steps, and the steps may be performed in any suitable order, as apparent to those skilled in the art.

To aid the Patent Office and readers in interpreting the claims, Applicants note that they do not intend any of the claims or claim elements to invoke 35 U.S.C. § 112 (f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the applicants to refer to structures known to those skilled in the relevant art and is not intended to invoke 35 U.S.C. § 112 (f).

What is claimed:

1. An ophthalmic system that tracks movement of an eye region, comprising:
a camera system comprising a plurality of cameras configured to yield a plurality of image portions of the eye region, each camera configured to image at least a part of the eye region to yield an image portion of the plurality of image portions, the camera system having a system axis and a system field of view, the eye region comprising one or both eyes, each eye of the eye region having an eye center and an eye axis; and
a computer configured to:
receive the plurality of image portions from the camera system; and
track movement of at least one eye of the eye region according to the plurality of image portions.

2. The ophthalmic system of claim 1, the computer configured to track the movement of at least one eye in two dimensions.

3. The ophthalmic system of claim 1, the computer configured to track the movement of at least one eye in three dimensions to allow for 6D tracking.

4. The ophthalmic system of claim 1, the plurality of cameras comprising a set of stereoscopic cameras arranged symmetrically about the system axis.

5. The ophthalmic system of claim 1, the plurality of cameras comprising a coaxial camera aligned with the system axis.

6. The ophthalmic system of claim 1, the plurality of cameras comprising an asymmetrically arranged camera, the asymmetrically arranged camera lacking a corresponding camera symmetrical about the system axis.

7. The ophthalmic system of claim 1, the plurality of cameras comprising a higher speed camera configured to generate images at greater than 400 frames per second.

8. The ophthalmic system of claim 1, the plurality of cameras comprising a higher resolution camera configured to generate images with greater than 4 megapixels.

9. The ophthalmic system of claim 1, at least one camera configured to detect a range of visible light from the eye region to yield an image portion.

10. The ophthalmic system of claim 1, at least one camera configured to detect a range of infrared light from the eye region to yield an image portion.

11. The ophthalmic system of claim 1, at least one camera configured to detect a range of ultraviolet light from the eye region to yield an image portion.

12. The ophthalmic system of claim 1:
further comprising a light projector configured to direct a pattern of light towards at least one eye of the eye region; and
at least one camera configured to detect the pattern of light reflected by the at least one eye.

13. The ophthalmic system of claim 1, the computer configured to track movement of at least one eye of the eye region according to the plurality of image portions by:
align the plurality of image portions to yield a combined image of the eye region; and
track movement of the at least one eye of the eye region according to the combined image of the eye region.

14. A method for tracking movement of an eye region, comprising:
providing, by a camera system comprising a plurality of cameras, a plurality of image portions of the eye region, each camera configured to image at least a part of the eye region to yield an image portion of the plurality of image portions, the camera system having a system axis and a system field of view, the eye region comprising one or both eyes, each eye of the eye region having an eye center and an eye axis;
receiving, by a computer, the plurality of image portions from the camera system; and
tracking, by the computer, movement of at least one eye of the eye region according to the plurality of image portions.

15. The method of claim 14, further comprising:
tracking the movement of at least one eye in two dimensions.

16. The method of claim 14, further comprising:
tracking the movement of at least one eye in three dimensions to allow for 6D tracking.

17. The method of claim 14, further comprising:
generating images at greater than 400 frames per second.

18. The method of claim 14, further comprising:
generating images with greater than 4 megapixels.

19. The method of claim 14, further comprising:
directing, by a light projector, a pattern of light towards at least one eye of the eye region; and
detecting, by at least one camera, the pattern of light reflected by the at least one eye.
